(19) United States
(12) Patent Application Publication
(10) Pub. No.: US 2022/0033859 A1
(43) Pub. Date: Feb. 3, 2022

(54) INCREASED BIOACTIVITY OF BIOPROTECTIVE CULTURES AGAINST PATHOGENIC BACTERIA

(71) Applicant: Chr. Hansen A/S, Hoersholm (DK)

(72) Inventors: Birgitte YDE, Hoersholm (DK); Hans BISGAARD-FRANTZEN, Hoersholm (DK); Jacob KAYA, Hoersholm (DK)

(73) Assignee: Chr. Hansen A/S, Hoersholm (DK)

(21) Appl. No.: 17/297,936

(22) PCT Filed: Nov. 29, 2019
(86) PCT No.: PCT/EP2019/083116

§ 371 Date: May 27, 2021

(30) Foreign Application Priority Data
Nov. 30, 2018 (EP) 18209478.9

(51) Int. Cl. C12P 1/04; A01N 63/20; C12N 1/20; A23L 33/135; A23L 3/3571; A23B 4/22; A23C 19/11

(52) U.S. Cl. CPC C12P 1/04; A01N 63/20; C12N 1/20; A23Y 2220/25; A23L 3/3571; A23B 4/22; A23C 19/11; A23L 33/135

(57) ABSTRACT

The present invention relates to a process for obtaining a biomass composition of a bacterium strain with bactericidal activity which inhibits or kills various pathogenic bacteria.

### FIELD OF THE INVENTION

The present invention relates to a novel and improved process for obtaining a biomass composition (including single or multiple bacterial cells) of a bacterium strain which inhibits or kills, with bactericidal activity, against various pathogenic bacteria. The invention further relates to the obtained composition and the use of the composition in particular food manufacturing.

### BACKGROUND OF THE INVENTION

Food poisoning involving various pathogens along with the increasing concern about the preservation of processed food, have given rise to increasing awareness of the importance of food safety. In recent years there have been an increasing interest in the antimicrobial activity of bacteria particularly lactic acid bacteria. Among the known antimicrobial activity of bacteria are bacteriocins. Bacteriocins are according to IngoIf F. Nes in handbook of Biologically Active Peptides (second edition), 2013 defined as ribosomally synthesized antibacterial peptides/proteins that either kill or inhibit the growth of closely related bacteria. These bacteriocins are divided into two major classes: The Class I lantibiotics and the Class II non-modified bacteriocins, with the latter also being called the non-lantibiotics. The Class II bacteriocins, are divided into: (a) the anti-listeria, pediocin-like bacteriocins that have very similar amino acid sequences at their N-terminus, (b) the two-peptide bacteriocins whose activity depends on two different peptides, (c) the cyclic bacteriocins, and (d) the linear nonpediocin-like one-peptide (LINPLOP) bacteriocins. In addition, there is a group named leaderless bacteriocins because they are synthesized without an N-terminal leader peptide.

To use bacteriocins to preserve food products are known in the art.

WO 99/67287 relates the production of a spray dried bacteriocin lacticin powder for use as a food ingredient. During production the pH is adjusted to 6.3 to 6.7.

WO02055672 relates to the production of a bacteriosin producing Lactococcus lactis transconjugants that can be used as a starter culture to accelerate cheese ripening.

The use of bacteriocin producing cultures in food is of considerable advantage for food safety, it has been found that the amount of active bacteriocins obtained after end of fermentation can be lost during the downstream processing. There is thus a desire to increase the amount of active bacteriocins, obtained from the culture medium during and after fermentation, in the final product

It is therefore the aim of the present invention to provide a process whereby the amount of active bacteriocins present in the biomass after end of fermentation is increased compared to known processes.

### SUMMARY OF THE INVENTION

The present invention relates to a method for obtaining a biomass with inhibiting bacterial growth and/or bactericidal activity. Said biomass is a means for inhibiting or avoiding growth of bacteria in food products, in particular in raw or cooked processed meat and dairy products.

A first aspect the present invention relates to a process for obtaining a biomass which inhibits bacterial growth and/or with bactericidal activity comprising the steps of
a) Obtaining a culture medium by cultivating a bacteriocin producing strain in a growth medium,
b) Adjusting the pH of the culture medium to below 5
c) Optionally adding a flocculant,
d) Separating the biomass from the culture medium,
e) Optionally adding an excipient to the biomass;
f) Optionally adjusting the pH of the biomass to a pH in the range of 5.5 to 8.0,
g) Optionally pelletizing the biomass,
h) Optionally freezing and/or drying the biomass before and/or after g), and
i) Optionally making the dried biomass into powder.

A second aspect of the present invention relates to a composition obtainable by the process of the first aspect.

A third aspect of the present invention relates to the use of the composition obtainable by the process of the first aspect for treating a food product.

A fourth aspect of the present invention relates to the use of the composition obtainable by the process of the first aspect for treating a fermented food product.

A fifth aspect of the present invention relates to the use of the composition obtainable by the process of the first aspect for reducing the concentration of Listeria spp. in a fermented food product.

A sixth aspect of the present invention relates to the use of the composition obtainable by the process of the first aspect for reducing the concentration of Listeria spp. in a meat product.

### DEFINITIONS

In the present context, the term “microorganism” is used in its normal meaning. Thus, in its broadest meaning the term “microorganism” is intended to cover algae, protozoa, bacteria and fungi. Preferred microorganisms are bacteria and fungi, in particular bacteria, such as lactic acid bacteria.

As used herein, the term “lactic acid bacterium” designates a gram-positive, microaerophilic or anaerobic bacterium, which ferments sugars with the production of acids including lactic acid as the predominantly produced acid. The industrially most useful lactic acid bacteria are found within the order “Lactobacillales” which includes Lactococcus spp., Streptococcus spp., Lactobacillus spp., Leuconostoc spp., Pseudoleuconostoc spp., Pediococcus spp., Brevibacterium spp. and Enterococcus spp. These are frequently used as food cultures alone or in combination with other lactic acid bacteria.

Lactic acid bacteria, including bacteria of the species Lactobacillus sp. and Streptococcus thermophilus, are normally supplied as frozen or freeze-dried cultures for bulk starter propagation or as so-called “Direct Vat Set” (DVS) cultures, intended for direct inoculation into a fermentation vessel or vat for the production of a food product. Such lactic acid bacterial cultures are in general referred to as “starter cultures” or “starters”. Merging applications of lactic acid bacteria further includes bioprotection of consumable foods e.g. meat products. Here the bacteria are applied to the food product in order to prolong the durability and quality of the food product by inhibiting pathogenic bacteria.

Commonly used starter culture strains of lactic acid bacteria are generally divided into mesophilic organisms having optimum growth temperatures at about 30° C. and thermophilic organisms having optimum growth temperatures in the range of about 40 to about 45° C. Typical organisms belonging to the mesophilic group include Lactococcus lactis, Lactococcus lactis subsp. cremoris, Leuconostoc mesenteroides subsp. cremoris, Pediococcus pentosaceus, Lactococcus lactis subsp. lactis biovar. diacetylactis, Lactobacillus casei subsp. casei and Lactobacillus paracasei subsp. paracasei. Thermophilic lactic acid bacterial species include as examples Streptococcus thermophi-lus, Pediococcus acidilactici, Enterococcus faecium, Lactobacillus delbrueckii subsp. lactis, Lacto-bacillus helveticus, Lactobacillus delbrueckii subsp. bulgaricus and Lactobacillus acidophilus.

Also the anaerobic bacteria belonging to the genus Bifidobacterium including Bifidobacterium bifidum and Bifidobacterium longum are commonly used as starter cultures and are generally included in the group of lactic acid bacteria. Additionally, species of Propionibacterium are used as starter cultures, in particular in the manufacture of cheese. Additionally, organisms be-longing to the Brevibacterium genus are commonly used as food starter cultures.

The term “biomass” is the amount of living matter in a given habitat, expressed either as the weight of organisms per unit area or as the volume of organisms per unit volume of habitat.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “to inhibit” and “to be inhibiting” in relation to unwanted microorganisms mean for example that the growth or the number or the concentration of unwanted microorganisms, for example in food products and/or on the surface of food products comprising the antimicrobial composition, is lower than in food products and/or on the surface of food products which does not comprise such an antimicrobial composition.

### BRIEF DESCRIPTION OF THE FIGURES

FIG. 1 discloses an illustration of a method for testing bioactivity of bioprotective culture against pathogenic bacteria e.g. Listeria.

FIG. 2 discloses the cell counts of Lactobacillus curvatus cultures measured after production on the novel process described in example 1 and 2.

FIG. 3 discloses the Logarithmic cell count of Listeria inocua over the logarithmic cell count of Lactobacillus curvatus. Three dilution curves are pictured for three different pH adjustments of pH 4.5, 6.5 and 8.5, respectively.

FIG. 4 discloses the cell counts of Lactobacillus curvatus cultures measured after production on the novel process described in example 1 and 5.

FIG. 5 disclose the calculated IC50 values.

FIG. 6 discloses the Logarithmic cell count of Listeria inocua over the logarithmic cell count of Lactobacillus curvatus. Two dilution curves are pictured—reference pH 6.5 and biomass not adjusted back to pH 6.5 respectively.

### DETAILED DESCRIPTION OF THE INVENTION

The present invention relates to a novel and improved process for obtaining a biomass composition (including a single or multiple bacterial cells) of a bacterium strain which inhibits or kills, with bactericidal activity, against various pathogenic bacteria. The invention further relates to the obtained composition and the use of the composition in particular food manufacturing.

Current bioprotective products for food applications such as meat are sold to costumers based on cell count. Here the bioprotective cultures are added to the customer's product in order to preserve the food product by inhibiting pathogenic bacteria (e.g. listeria). This inhibitory effect is believed to derive from a bacteriocin production of the bioprotective culture.

The present invention is describing a method for increasing the inhibitory effect of the bioprotective culture, while decreasing the impact on the product's taste. Since the growth of the culture is undesired it is believed that bacteriocin production already has occurred during production of the bioprotective culture, where the bacteriocin has been released to the extracellular environment. By conventional production methods most of the extracellular bacteriocin would be lost during cell concentration (centrifugation, microfiltration, etc.) in the eluate. This invention increases the amount of bacteriocin in the biomass by lowering the pH after end of cultivation. Without being bound by theory the bacteriocin is believed to aggregate and/or precipitate and thereby it can be trapped in the biomass. After biomass separation the pH value may be increased again if needed to retain the potency of the culture.

The present culture medium is obtained by cultivating a bacteriocin producing strain in a growth medium.

Suitable strains may be any strains producing bacteriocin. Preferred strains belong to Lactic acid bacteria (LAB), Leuconostoc carnosum, Lactobacillus species, such as Lactobacillus curvatus, Lactobacillus reuteri, Lactobacillus delbrueckii, Lactobacillus salivarius, Lactobacillus plantarum, Lactococcus lactis and Pediococcus species such as Pediococcus pentosaceus and Pediococcus acidilactici. In particular the Lactobacillus curvatus strain CHCC26906 (DSM 32591) and the Lactobacillus curvatus strain CHCC23218 (DSM 32590). It is how-ever contemplated that other bacteriocin-producing species may provide the same advantageous characteristics and effects as those illustrated herein.

The growth medium may be any suitable growth medium i.e. MRS media.

According to the invention the pH of the culture medium is adjusted to a pH below 5 after finalized cultivation. In a particular embodiment the pH is adjusted to a pH below 4.5, such as below 4, such as below 3.5, such as below 3 after finalized cultivation. Normally the pH adjustment will happen after end of fermentation/cultivation. End of cultivation is once the parameter determining the end of fermentation/cultivation has been reached e.g. when all consumable sugars has depleted, a concentration of a metabolite has been produced, time criteria, stop of base/acid addition, optical density criteria, etc.

The adjustment of pH may be performed with any suitable acid.

A flocculant may be added to the obtained biomass.

After adjusting pH to below 5 the biomass is separated from the culture medium. The selected method for separation may be any suitable method known in the art. In a particular embodiment of the present invention the separation step is performed by centrifugation or filtration i.e. microfiltration.

After separation the pH of the biomass may be adjusted to a pH above 5. In a particular embodiment of the present invention the pH is adjusted to a pH above 5, such as to a pH of 5.5 to 9, such as to a pH of 5.5 to 8.

The process can be performed at a temperature in the range 0 to 50° C., such as in the ranges 5 to 30° C. or 15 to 25° C. In a particular embodiment of the present invention the process is performed at ambient temperature.

The present invention relates to a process for obtaining a biomass which inhibits bacterial growth and/or with bactericidal activity comprising the steps of
a) Obtaining a culture medium by cultivating a bacteriocin producing strain in a growth medium,
b) Adjusting the pH of the culture medium to below 5,
c) Optionally adding a flocculant,
d) Separating the biomass from the culture medium,
e) Optionally adding an excipient to the biomass; and/or optionally adjusting the pH of the biomass to a pH in the range of 5.5 to 8.0,
f) Optionally pelletizing the biomass, and
g) Optionally freezing and/or drying the biomass before and/or after f.

Separation in step c) may be performed by any suitable method known in the art. In a particular embodiment of the present invention the separation step is performed by centrifugation or filtration i.e. microfiltration.

The biomass is preferably pelletized, granulated or made into a powder.

The biomass is preferably frozen and/or dried i.e. by freeze drying or spray drying by conventional techniques known in the art.

The present invention relates to a process for obtaining a biomass which inhibits bacterial growth and/or with bactericidal activity comprising the steps of
a) Obtaining a culture medium by cultivating a bacteriocin producing strain in a growth medium,
b) Adjusting the pH of the culture medium to below 5,
c) Optionally adding a flocculant,
d) Separating the biomass from the culture medium,
e) Optionally adding an excipient to the biomass; and/or optionally adjusting the pH of the biomass to a pH in the range of 5.5 to 8.0,
f) Optionally pelletizing the biomass, and
g) Optionally freezing and/or drying the biomass before or after f.

Separation in step c) may be performed by any suitable method known in the art. In a particular embodiment of the present invention the separation step is performed by centrifugation or filtration i.e. microfiltration.

In a particular embodiment of the present invention relates to a process for obtaining a biomass which inhibits bacterial growth and/or with bactericidal activity comprising the steps of
a) Obtaining a culture medium by cultivating a bacteriocin producing strain in a growth medium,
b) Adjusting the pH of the culture medium to below 5,
c) Separating the biomass from the culture medium, and
d) Adding an excipient to the biomass.

Excipients may be added at any time during the process. In a particular embodiment of the present invention the excipients are added after separation. The excipients may be any suitable excipients known in the art i.e. cryo protectants such as monosaccharides, disaccharides, oligosaccharides, polysaccharides and antioxidants. Particular protectants may be starch hydrolysates (e.g. dextrin from maize starch), sodium glutamate, polyol (e.g. mannitol, sorbitol).

In a particular embodiment the present invention relates to a process for obtaining a biomass which inhibits bacterial and/or with bactericidal activity comprising the steps of
a) Obtaining a culture medium by cultivating a bacteriocin producing strain in a growth medium,
b) Adjusting the pH of the culture medium to below 5,
c) Separating the biomass from the culture medium, and
d) Adjusting the pH of the biomass to a pH in the range of 5.5 to 8.0.

In a particular embodiment of the present invention relates to process for obtaining a biomass which inhibits bacterial growth and/or with bactericidal activity comprising the steps of
a) Obtaining a culture medium by cultivating a bacteriocin producing strain in a growth medium,
b) Adjusting the pH of the culture medium to below 5,
c) Separating the biomass from the culture medium, and
d) Pelletizing the biomass or granulating the biomass or making it into a powder.

In a particular embodiment of the present invention pelletizing of the biomass is performed by use of liquid nitrogen.

The present invention relates to a process for obtaining a biomass which inhibits bacterial and/or with bactericidal activity comprising the steps of
a) Obtaining a culture medium by cultivating a bacteriocin producing strain in a growth medium,
b) Adjusting the pH of the culture medium to below 5,
c) Separating the biomass from the culture medium, and
d) Freezing and/or drying the biomass.

The present invention relates to a process for obtaining a biomass which inhibits bacterial and/or with bactericidal activity comprising the steps of
a) Obtaining a culture medium by cultivating a bacteriocin producing strain in a growth medium,
b) Adjusting the pH of the culture medium to below 5,
c) Separating the biomass from the culture medium,
d) Optionally adding an excipient to the biomass; and/or optionally adjusting the pH of the biomass to a pH in the range of 5.5 to 8.0,
e) Optionally pelletizing the biomass, and
f) Optionally freezing and/or drying the biomass.

The present invention further relates to a composition obtainable or obtained by the process of the present invention.

In a particular embodiment of the present invention the inhibitory effect, of the bioprotective culture, against listeria is increased by at least 90%, which is equivalent to a lower inoculation of 0.9 LOG unit of the bioprotective culture compared to the current inoculation level.

The composition of the present invention is comprising a biomass comprising a viable bacteriocin producing strain and bacteriocin.

The present invention further relates to the use of the composition obtainable or obtained by the process of the present invention for treating a food product.

The foods most often associated with contamination by Listeria monocytogenes are milk based products such as milk based cheeses, ice cream and Cottage cheese, processed vegetables, smoked food products, meat and meat based products. Foods that are handled by machinery and are not heat-treated in final package are particularly vulnerable. Meats, such as beef, pork or poultry, can be contaminated during or after slaughtering. Fish can also be contaminated in processing.

In a particular embodiment of the present invention the present invention relates to the use of the composition obtainable or obtained by the process of the first aspect for treating a fermented food product.

In a particular embodiment of the present invention the invention relates to the use of the composition obtainable obtained by the process of the present invention for reducing the concentration of a pathogenic organism such as Listeria spp. in a fermented food product.

In a particular embodiment of the present invention the invention relates to the use of the composition obtainable or obtained by the process of the present invention for reducing the concentration of a pathogenic organism such as Listeria spp. in a meat product.

In a particular embodiment of the present invention the present invention relates to the use of the composition obtainable or obtained by the process of the resent invention in probiotic products.

In the present context the term “reducing the concentration” relates to a reduction in the amount of a pathogenic organism. A reduction may be provided by killing, inactivating or inhibiting the activity of the pathogenic organism. In an embodiment of the present invention 100% of the pathogenic organism are killed, inactivated or inhibited, such as at least 90%, e.g. at least 75%, such as at least 50%, e.g. at least 40%, such as at least 30%, e.g. at least 25%, such as at least 20%, e.g. at least 10%, such as at least 5%, e.g. at least 1%.

In certain applications, an inhibition of the pathogenic organisms that may be present in the food will be sufficient to render the food safe. Thus, the culture secures that the pathogenic organisms that are present in the food do not increase in number.

In a particular embodiment the present invention relates to a method comprising the steps of:
a) providing a food material,
b) mixing the food material with the composition of the present invention.

In further detail, the present invention relates to the following aspects:
Aspect 1. A process for obtaining a biomass which inhibits bacterial and/or fungal growth and/or with bactericidal and/or fungicidal activity comprising the steps of
   a) Obtaining a culture medium by cultivating a bacteriocin producing strain in a growth medium,
   b) Adjusting the pH of the culture medium to below 6,
   c) Optionally adding a flocculant,
   d) Separating the biomass from the culture medium,
   e) Optionally adding an excipient to the biomass
   f) Optionally adjusting the pH of the biomass to a pH in the range of 6 to 9.0,
   g) Optionally pelletizing the biomass,
   h) Optionally freezing and/or drying the biomass before or after g), and
   i) Optionally making the dried biomass into powder.
Aspect 2. The process of aspect 1, wherein b) is performed after finalized cultivation.
Aspect 3. The process according to the preceding aspect, where the pH is kept below 6 for at least 1 hour.
Aspect 4. The process according to aspect 1, wherein the pH of the culture medium in b) is adjusted to below 5.5.
Aspect 5. The process according to aspect 1, wherein the pH of the culture medium in b) is adjusted to below 5.
Aspect 6. The process according to the preceding aspect, where the pH is kept below 5 for at least 1 hour.
Aspect 7. The process according to aspect 1, wherein the pH of the culture medium in b) is adjusted to below 4.8.
Aspect 8. The process according to aspect 1, wherein pH of the culture medium in b) is adjusted to between 2.0 and 5.0.
Aspect 9. The process according to any of the preceding aspects, wherein the separation in c) is performed by centrifugation and/or filtration.
Aspect 10. The process according to any of the preceding aspects, wherein the bacterial strain belongs to a Lactobacillus species or a Pediococcus species.
Aspect 11. The process according to aspect 10, wherein the bacterial strain is selected from the group consisting of, Leuconostoc carnosum, Lactobacillus curvatus, Lactobacillus reuteri, Lactobacillus delbrueckii, Lactobacillus salivarius, Lactobacillus plantarum, Lactococcus lactis, Pediococcus pentosaceus, Pediococcus acidilactici.
Aspect 12. The process according to any of the preceding aspects, wherein a flocculant is added after adjusting the pH of the culture medium to below 6.
Aspect 13. The process according to any of the preceding aspects, wherein a flocculant is added after adjusting the pH of the culture medium to below 5.
Aspect 14. The process according to any of the preceding aspects, wherein an excipient is added to the biomass after it has been separated from the culture medium.
Aspect 15. The process according to any of the preceding aspects, wherein the pH of the biomass is adjusted to between 5.5 to 8.0.
Aspect 16. The process according to any of the preceding aspects, wherein the pH of the biomass is adjusted to between 6.5 to 9.0.
Aspect 17. The process according to any of the preceding aspects, wherein the pH of the biomass is adjusted to between 6.5 to 8.0.
Aspect 18. The process according to any of the preceding aspects, wherein the biomass is pelletized into pellets, granulated into granules or made into a powder.
Aspect 19. The process according to any of the preceding aspects, wherein the pellets, granules or powder of aspect 18 are frozen and/or dried.
Aspect 20. A composition obtainable by a process according to any of the preceding aspects comprising a bacteriocin producing strain and a bacteriocin.
Aspect 21. Use of the composition of aspect 20 for treating a food product.
Aspect 22. The use of aspect 21, wherein the food product is a dairy product or a meat product.

### DEPOSIT AND EXPERT SOLUTION

The applicant requests that a sample of the deposited microorganisms stated below may only be made available to an expert, until the date on which the patent is granted.

The Lactobacillus curvatus strain CHCC26906 was deposited 16 Aug. 2017 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 32591.

The Lactobacillus curvatus strain CHCC23218 was deposited 16 Aug. 2017 at German Collection of Microorganisms and Cell Cultures (Deutsche Sammlung von Mikroorganismen and Zellkulturen GmbH; DSMZ), Inhoffenstr. 7B, D-38124 Braunschweig and given the accession No.: DSM 32590.

### EXAMPLES

### Example 1. Obtaining Biomass of Lactobacillus curvatus

Lactobacillus curvatus culture was grown in a typical growth media comprising of, in w/v percentages, 1.0% peptone from casein, 1.0% meat extract, 0.4% yeast extract, 2.0% glucose, 0.5% sodium acetate trihydrate, 0.1% polysorbate 80, 0.2% dipotassium hydrogen phosphate, 0.2% tri-ammonium citrate, 0.02% magnesium sulphate heptahydrate and 0.005% manganese sulphate tetrahydrate. The fermentation was carried out in 350 L scale at ambient temperature, stirring speed of 300 rpm and at 6.5 pH.

### Example 2. Harvesting High Active Biomass from Lactobaccillus Culture with pH Adjustment to 4.5

At end of fermentation 2 L of culture medium was transferred to glass beakers. The pH of the culture medium was adjusted from 6.5 pH (example 1) to pH 4.5 with a solution of phosphoric acid. The culture was kept for 1 hour at room temperature with slow stirring at 50 rpm. After 1 hour holding time the biomass was separated from the culture medium by centrifugation at 4200 rpm for 20 min. After centrifugation the biomass concentrate was collected in a suitable sized glass beaker and the pH was adjusted from 4.5 to 6.5 with a solution of sodium hydroxide during slow stirring of 50 rpm. Cryo-protective solution (which consisted of sucrose (15%), maltodextrin (10%) and water (75%)) was added (420 g to 1000 g cell concentrate) to the concentrate. Finally, the biomass concentrate was pelletized in fluid nitrogen and freeze dried.

### Example 3. Harvesting High Active Biomass from Lactobaccillus Culture with pH Adjustment to 5.0

At end of fermentation 2 L of culture medium was transferred to glass beakers. The pH of the culture medium was adjusted from 6.5 pH (example 1) to pH 5.0 with a solution of phosphoric acid. The culture was kept for 1 hour at room temperature with slow stirring at 50 rpm. After 1 hour holding time the biomass was separated from the culture medium by centrifugation at 4200 rpm for 20 min. After centrifugation the biomass concentrate was collected in a suitable sized glass beaker and the pH was adjusted from 5.0 to 6.5 with a solution of sodium hydroxide during slow stirring of 50 rpm. Cryo-protective solution (which consisted of sucrose (15%), maltodextrin (10%) and water (75%)) was added (420 g to 1000 g cell concentrate) to the concentrate. Finally, the biomass concentrate was pelletized in fluid nitrogen and freeze dried.

### Example 4. Harvesting High Active Biomass From Lactobaccillus Culture With pH Adjustment to 3.5

At end of fermentation 2 L of culture medium was transferred to glass beakers. The pH of the culture medium was adjusted from 6.5 pH (example 1) to pH 3.5 with a solution of phosphoric acid. The culture was kept for 1 hour at room temperature with slow stirring at 50 rpm. After 1 hour holding time the biomass was separated from the culture medium by centrifugation at 4200 rpm for 20 min. After centrifugation the biomass concentrate was collected in a suitable sized glass beaker and the pH was adjusted from 3.5 to 6.5 with a solution of sodium hydroxide during slow stirring of 50 rpm. Cryo-protective solution (which consisted of sucrose (15%), maltodextrin (10%) and water (75%)) was added (420 g to 1000 g cell concentrate) to the concentrate. Finally, the biomass concentrate was pelletized in fluid nitrogen and freeze dried.

### Example 5. Harvesting High Active Biomass from Lactobaccillus Culture Maintaining pH Of Biomass at pH 4.5

At end of fermentation 2 L of culture medium was transferred to glass beakers. The pH of the culture medium was adjusted from 6.5 pH (example 1) to pH 4.5 with a solution of phosphoric acid. The culture was kept for 1 hour at room temperature with slow stirring at 50 rpm. After 1 hour holding time the biomass was separated from the culture medium by centrifugation at 4200 rpm for 20 min. After centrifugation the biomass concentrate was collected in a suitable sized glass beaker. Cryo-protective solution (which consisted of sucrose (15%), maltodextrin (10%) and water (75%)) was added (420 g to 1000 g cell concentrate) to the concentrate. Finally, the biomass concentrate was pelletized in fluid nitrogen and freeze dried.

### Example 6. Testing Activity Against Listeria innocua

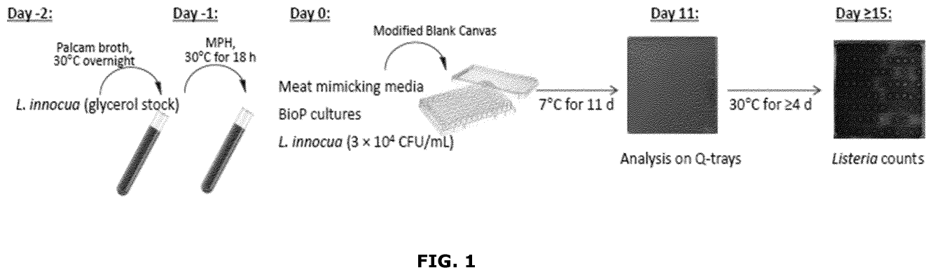

The activity of the Lactobacillus cultures was tested against Listeria in a co-cultivational method. Listeria was grown in an overnight culture in Palcom broth at 30 degrees celsius and then transferred to a meat mimicking media (meat pH media, MPH) at 30 degrees celsius for 18 hours. Hereafter the Listeria culture and the Lactobacillus culture were co-cultivated in a meat mimicking media at 7 degrees celsius for 11 days. Both Listeria and Lactobacillus cultures were inoculated at a fixed CFU/g cell count. Finally, the Listeria was analyzed for CFU/g cell counts. The method is summarized in FIG. 1.

### Example 7. Harvesting High Active Biomass from Lactobaccillus Culture

A sample was obtained according to the procedures described in example 1 and 2, respectively. This sample was tested in combination with a sample with no pH adjustment (pH 6.5) after end of fermentation and a sample adjusted to pH 8.5 (with a solution of sodium hydroxide) at end of fermentation and then continuously processed as in example 2.

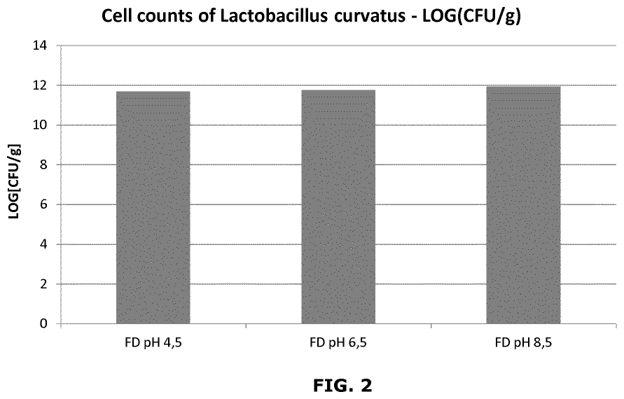

The CFU/g cell counts of the freeze dried samples were analyzed. Results can be found in FIG. 2.

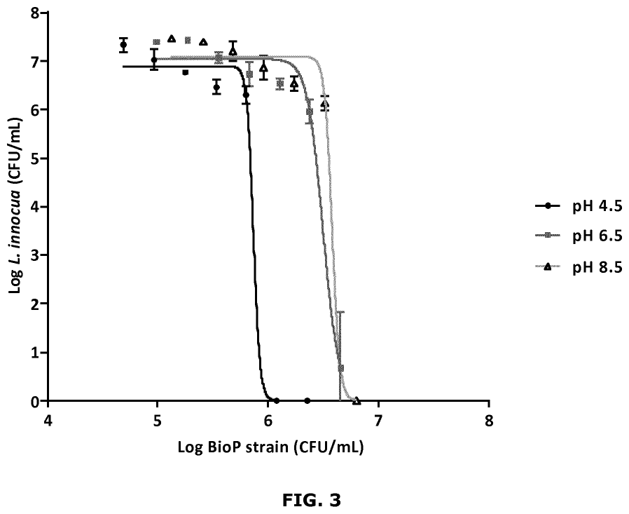

The samples were tested with the method described in example 6. This resulted in the data illustrated in FIG. 3. Here a dilution curve was prepared by testing 7 different dilution levels of the bioactive culture against Listeria for each of the 3 samples (pH 4.5, pH 6.5, pH 8.5). Hereafter the CFU of Listeria were measured. From FIG. 3 it can be seen that at a certain concentration no Listeria cells were counted on the CFU method. Furthermore, it can be seen that for the pH 4.5 treated sample (Example 2) the Listeria cells are inhibited at a lower concentration than the pH 6.5 (Example and 8.5 samples.

### Example 8. Testing the Effect of pH Readjustment from 4.5 to 6.5

In another case a sample was obtained according to the procedure described in example 1 (pH 4.5) and Example 5 (ph 4.5 without pH adjustment to pH 6.5). Again this sample was tested in combination with a sample with no pH adjustment (pH 6.5) after end of fermentation.

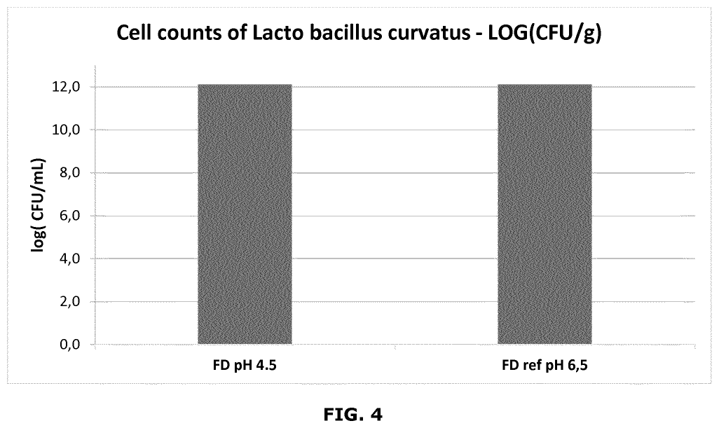

The CFU/g cell counts of the freeze dried samples were analyzed. Results can be found in FIG. 4. The results confirm that the treatments result in comparable CFU levels.

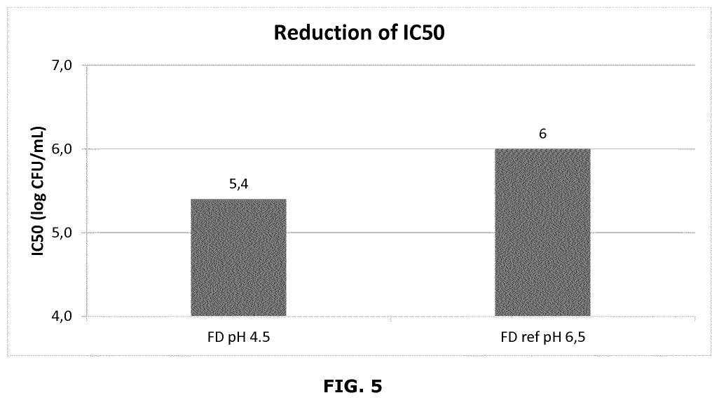

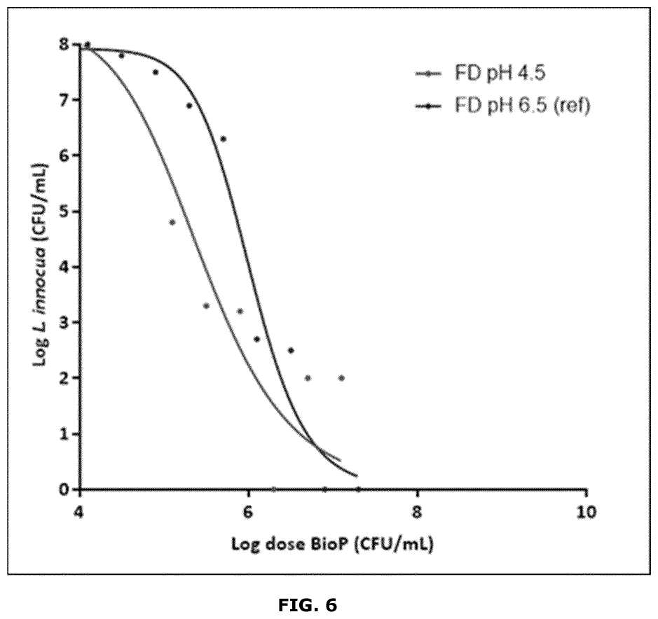

The samples were tested with the method described in example 6. This resulted in the data illustrated in FIGS. 5 and 6. Here a dilution curve was prepared by testing 7 different dilution levels of the bioactive culture against Listeria for each of the 2 samples (pH 4.5 where the biomass was not adjusted back to pH 6.5 and pH 6.5 reference). Hereafter the CFU of Listeria were measured. From FIGS. 5 and 6 it is evident that for non pH adjusted sample the Listeria cells are inhibited at a lower concentration than the reference samples equal to a reduction in the IC 50 value of 0.6 log units.

1. A process for obtaining a biomass with one or both of bactericidal and fungicidal activity, comprising:
a) cultivating a bacteriocin-producing strain in a growth medium to obtain a biomass in a culture medium,
b) adjusting the pH of the culture medium to below 5, and
c) separating the biomass from the culture medium.

2. The process of claim 1, wherein step (b) is performed after the end of cultivation.

3. The process according to claim 2, wherein the pH is kept below 5 for at least 1 hour.

4. The process according to claim 1, wherein step (b) comprises adjusting the pH of the culture medium to below 4.8.

5. The process according to claim 1, wherein step (b) comprises adjusting the pH of the culture medium to between 2.0 and 5.0.

6. The process according to claim 1, wherein step (c) comprises one or both of centrifugation and filtration.

7. The process according to claim 1, wherein the bacterial strain belongs to a Lactobacillus species or a Pediococcus species.

8. The process according to claim 7, wherein the bacterial strain is of a species selected from the group consisting of Leuconostoc carnosum, Lactobacillus curvatus, Lactobacillus reuteri, Lactobacillus delbrueckii, Lactobacillus salivarius, Lactobacillus plantarum, Lactococcus lactis, Pediococcus pentosaceus, and Pediococcus acidilactici.

9. The process according to claim 1, further comprising adding a flocculant to the culture medium after step (b) of adjusting the pH of the culture medium to below 5.

10. The process according to claim 1, further comprising adding an excipient to the biomass after step (c) of separating the biomass from the culture medium.

11. The process according to claim 1, further comprising adjusting the pH of the biomass to between 5.5 and 8.0.

12. The process according to any claim 1, further comprising pelletizing the biomass into pellets, granulating the biomass into granules, or making the biomass into a powder.

13. The process according to claim 12, further comprising one or both of freezing and drying the pellets, granules or powder.

14. A composition obtained by a process according to claim 1, comprising the biomass comprising the bacteriocin-producing strain and a bacteriocin.

15. A method for treating a food product, comprising contacting the food product with a biomass according to claim 14.

16. The method of claim 15, wherein the food product is a dairy product or a meat product.

17. The process according to claim 11, wherein the pH of the biomass is adjusted to pH 6.5.

18. The process according to claim 1, further comprising one or both of freezing and drying the biomass.

19. The process according to claim 1, further comprising drying the biomass and making the dried biomass into a powder.
